(19) United States
(12) Patent Application Publication
(10) Pub. No.: US 2026/0152777 A1
(43) Pub. Date: Jun. 4, 2026

(54) METHOD FOR PRODUCING EPIGALLOCATECHIN GALLATE MONOGLUCOSIDE

(71) Applicant: GIVAUDAN SA, VERNIER (CH)

(72) Inventors: Daniel AURIOL, Toulouse (FR); Cloe BOIRA, Pomacle (FR); Eglantine DON SIMONI, Toulouse (FR)

(21) Appl. No.: 19/124,163

(22) PCT Filed: Nov. 8, 2023
(86) PCT No.: PCT/EP2023/081192

§ 371 Date: Apr. 24, 2025

(30) Foreign Application Priority Data
Nov. 8, 2022 (GB) 2216603.7

(51) Int. Cl. C12P 19/46; A61K 8/60; A61Q 19/02; A61Q 19/08; C12N 9/10

(52) U.S. Cl. CPC C12P 19/46; A61K 8/602; A61Q 19/02; A61Q 19/08; C12N 9/1051; C12Y 204/01005

(57) ABSTRACT

Methods for selectively preparing monoglucosides of epigallocatechin and epigallocatechin gallate are provided, and the use of these products in cosmetics.

The present invention relates to methods of selectively preparing monoglucosides of epigallocatechin and epigallocatechin gallate, and to their use in cosmetics.

Phenolic compounds (also called phenolics), or polyphenols, constitute one of the most numerous and widely-distributed groups of substances in the plant kingdom. Polyphenols are products of the secondary metabolism of plants. The expression “phenolic compounds” embraces a considerable range of substances that possess an aromatic ring bearing one or more hydroxyl substituents.

The structure of natural polyphenols varies from simple molecules, such as phenolic acids, to highly polymerized compounds, such as condensed tannins.

The three important groups for humans are phenolic acids (C6-C1, C6-C2 and C6-C3), flavonoids (C6-C3-C6) and high-molecular weight polyphenols (more than 30 carbon atoms). Indeed, the phenolics, particularly polyphenols, exhibit a wide variety of beneficial biological activities in mammals, including antiviral, antibacterial, immune-stimulating, antiallergic, antihypertensive, antiischemic, antiarrhytmic, antithrombotic, hypocholesterolemic, antilipoperoxidant, hepatoprotective, anti-inflammatory, anticarcinogenic antimutagenic, antineoplastic, antithrombotic, and vasodilatory actions. They are powerful antioxidants in vitro.

The flavonoids consist of a large group of low-molecular weight polyphenols substances, benzo-β-pyrone derivatives that are diverse in chemical structure; they represent the most common and widely distributed group of plant phenolics. The flavonoids common structure is that of diphenylpropanes (C6-C3-C6), which consists of two aromatic rings (cycles A and B) linked through three carbons that usually form an oxygenated heterocycle (cycle C). The basic structure and the system used for the carbon numbering of the flavonoid nucleus is shown below:

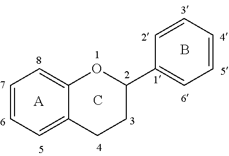

Flavonoids often occur as glycosides, glycosylation rendering the molecule more water-soluble and less reactive toward free radicals. Flavonoids have an ideal structural chemistry for free radical-scavenging activities.

Among the flavonoids, epigallocatechin (1) and epigallocatechin gallate (EGCG) (2) are of particular interest for cosmetic applications because of their anti-oxidant, anti-inflammatory, anti-microbial, anti-allergenic, anti-viral, and anti-pigmentation activity, as well as their ability to provide UV protection, activate the skin barrier, and promote cell activity.

The structures of these two flavonoids are shown below:

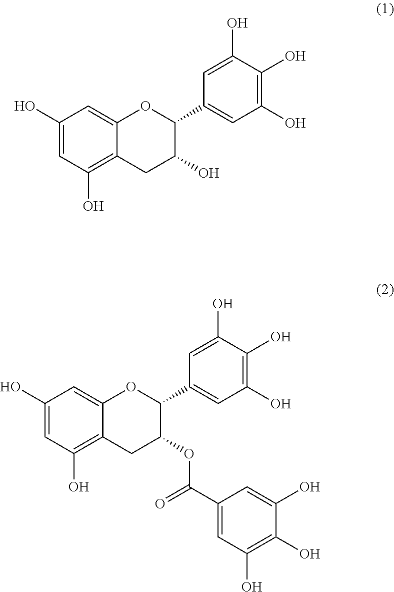 [SMILES: Oc1cc(O)c2c(c1)O[C@H](c1cc(O)c(O)c(O)c1)[C@H](O)C2 ; O=C(O[C@@H]1Cc2c(O)cc(O)cc2O[C@@H]1c1cc(O)c(O)c(O)c1)c1cc(O)c(O)c(O)c1]

Due to their low aqueous solubility and/or high sensitivity toward oxidation, the use of phenolics in pharmaceutical or cosmetic preparations requires adapted and specific formulations. Since these formulations must also satisfy the constraints associated with their final usage, the compromise between acceptability, concentration and stability is often difficult to reach. More water soluble and/or oxidation resistant forms of phenolics, such as the glycosides, are not always available in nature and may demand, when they exist, complex procedures of extraction and purification from the plant material. Both chemical and biochemical (enzymatic) approaches have been attempted to increase water solubility and/or stability. As phenolic compounds have several free hydroxyl groups, attempts for chemical modifications of phenolic compounds lead to unselective reactions, generating a variety of different molecules. Further steps of purification are then required to recover the desired product(s).

WO 2007/144368 discloses enzymatic methods for producing O-□-glucosides of phenolics using a glucansucrase from Leuconostoc species. Among others, epigallocatechin O-□□-glucoside and epigallocatechin gallate O-□□-glucoside are formed. The enzymatic reaction is typically conducted in a buffered aqueous medium comprising dimethyl sulfoxide (DMSO) as a co-solvent.

The method described in WO 2007/144368 affords epigallocatechin O-□□-glucoside and epigallocatechin gallate O-□□-glucoside, respectively, as a mixture of mono-, di- and tri-glucosides and with a relatively low regioselectivity. Typically, the mono- and di-glucosides are obtained in a molar ratio of about 30:70 to 60:40.

It is therefore a problem of the present invention to provide a method for preparing epigallocatechin O-□□-glucoside and epigallocatechin gallate O-□□-glucoside in a more selective way, without compromising on yield nor scalability.

This problem has been solved by the method of the present invention as described below.

Surprisingly, it has been found that the epigallocatechin gallate 4′-O-α-monoglucoside and the epigallocatechin 4′-O-α-monoglucoside, respectively, which have been selectively prepared by the method of the present invention, exhibit an improved skin penetration and bioaccumulation. They are therefore better suitable for use in cosmetic applications, in particular in skin care applications.

In a first aspect, the present invention provides a method of preparing epigallocatechin gallate 4′-O-α-monoglucoside.

In a second aspect, the present invention provides a method of preparing epigallocatechin 4′-O-α-monoglucoside.

In a third aspect, the present invention provides epigallocatechin gallate 4′-O-α-monoglucoside and epigallocatechin 4′-O-α-monoglucoside, respectively, prepared by the methods of the present invention.

In a fourth aspect, the present invention provides a method for providing a radiant skin complexion and/or age-spot removal, wherein epigallocatechin gallate 4′-O-α-monoglucoside is topically applied to the skin.

The present invention relates to a method of preparing epigallocatechin gallate 4′-O-α-monoglucoside comprising incubating sucrose and a glucansucrase from Leuconostoc species with epigallocatechin gallate. The incubation is conducted in a medium comprising 2-propanol and water.

Surprisingly, it has been found that by using 2-propanol as a co-solvent in the medium, the selectivity of the enzymatic reaction can be significantly improved, selectively affording the monoglucoside and with high regioselectivity. In particular, the observed regioselectivity was better than if a diol or triol was used as the co-solvent.

The method of the present invention can be carried out at concentrations suitable for an industrial production (e.g. about 15 to 30 mM) and affords epigallocatechin gallate 4′-O-α-monoglucoside in a yield of more than 30%. Preferred reaction conditions are indicated further down.

For comparison, under otherwise identical conditions:
If other short chain alcohols were used as the co-solvent, much lower conversions were observed (ethanol: 8%, 1-propanol: 0.71%, 1-butanol: 0.08%, and 2-butanol: 0.25%; vs. 48.5% conversion for 2-propanol).
If only 1,3-propanediol was used as the co-solvent, a conversion of 60% was achieved, 45% being the monoglucoside (58% of which is the 4′-O-α-monoglucoside), 14% the diglucoside and 1% the triglucoside.
If only 2,3-butanediol was used as the co-solvent, a conversion of 57% is achieved, 45% being the monoglucoside (66% of which was the 4′-O-α-monoglucoside), 14% the diglucoside and 1% the triglucoside.
If only 1,3-butanediol was used as the co-solvent, a conversion of 67% is achieved, 47% being the monoglucoside (65% of which was the 4′-O-α-monoglucoside), 19% the diglucoside and 1% the triglucoside.
If only 2,4-butanediol was used as the co-solvent, a conversion of 64% is achieved, 44% being the monoglucoside (62% of which was the 4′-O-α-monoglucoside), 19% the diglucoside and 1% the triglucoside.
With DMSO as the co-solvent, a conversion of 61% was achieved, 40% being the monoglucoside (63% of which was the 4′-O-α-monoglucoside), 19% the diglucoside and 2% the triglucoside.
If no co-solvent was used, no conversion is observed.

The present invention further relates to a method of preparing epigallocatechin 4′-O-α-monoglucoside comprising incubating sucrose and a glucansucrase from Leuconostoc species with epigallocatechin. The incubation is conducted in a medium comprising 2-propanol and water.

Again, the use of 2-propanol as a co-solvent in the medium has been found to improve the selectivity of the enzymatic reaction, in particular the regioselectivity of the glucosylation. In addition, the enzyme efficacy was significantly better than with other short chain alcohols.

The enzymatic reaction is achieved using sucrose, an abundant and rather cheap substance used in the food and feed fields. This reaction consists in the transfer of the glucose part of sucrose on a hydroxyl group of the catechol ring. Once a first glucosyl residue has been attached to a hydroxyl group of the catechol ring, a further transfer of the glucose part of sucrose may occur, either to another hydroxyl group of the catechol ring or to a hydroxyl group of the fixed glucose, affording a di- or even tri-glucoside.

The methods of the present invention allow for significantly reducing or even completely avoiding the formation of di- and tri-glucosides, and for obtaining the mono-glucosides in high regioselectivity.

In the methods of the present invention, a glucansucrase from Leuconostoc species is used. WO 2007/144368 mentions several suitable glucansucrases, which are herewith incorporated by reference.

In a preferred embodiment, a glucansucrase from Leuconostoc mesenteroides NRRL B-512F (ATCC 10830a) is used.

The methods of the present invention are carried out in an aqueous medium. Said aqueous medium is preferably buffered at a pH convenient for the enzymatic activity (well known by a skilled person).

The nature of buffer used in the medium is not very important. However, preferably, buffering agents that are able to chelate divalent cations, such as Ca²⁺ or Mg²⁺, should be avoided. Suitable buffers include, but are not limited to, acetate buffers (e.g. sodium or potassium acetate) or sodium propionate buffer.

In an embodiment, a sodium or potassium acetate buffer is used, preferably at a concentration ranging from 5 to 100 mM, more preferably from 10 to 50 mM and even more preferably from 15 to 40 mM.

In an embodiment, the pH of the buffer is from 5.00 to 5.60, more preferably from 5.10 to 5.50, and even more preferably from 5.20 to 5.40.

In the methods of the present inventions, the incubation is conducted in a medium comprising 2-propanol and water.

In an embodiment, the medium comprises 5% to 20% (v/v) of 2-propanol, more preferably 8% to 18% (v/v). These concentration ranges have been found to prevent enzyme denaturation under the reaction conditions, thereby providing the desired products in high yield even at relatively low enzyme concentrations.

Optionally, the medium may further comprise a lower diol and/or a lower triol, in particular a lower diol and/or a lower triol selected from the group consisting of 1,3-propanediol, 1,2-propanediol, 1,2-butanediol, 1,3-butanediol, 1,4-butanediol, 2,3-butanediol, 2,4-butanediol, 1,2,3-propanetriol (glycerol), and mixtures thereof. Surprisingly, it has been found that the regioselectivity of the reaction is even better if a combination of 2-propanol and 1,3-propanediol is used.

The medium used in the methods of the present invention should not comprise any co-solvents or other additives that inhibit the enzyme reactivity and/or reduce the selectivity.

In an embodiment, the medium does not comprise any dimethylsulfoxide.

In an embodiment, the medium does not comprise any ethers of ethylene glycol (glymes).

The epigallocatechin gallate 4′-O-α-monoglucoside and the epigallocatechin 4′-O-α-monoglucoside, respectively, are obtained by incubating the respective substrates with sucrose and a glucansucrase.

In an embodiment, the epigallocatechin gallate is incubated at an initial concentration of from 5 to 50 mM, more preferably from 10 to 40 mM, and most preferably from 15 to 35 mM, to obtain epigallocatechin gallate 4′-O-α-monoglucoside.

Alternatively, in order to obtain epigallocatechin 4′-O-α-monoglucoside, epigallocatechin is incubated at an initial concentration of from 5 to 50 mM, more preferably from 10 to 40 mM, and most preferably from 15 to 35 mM.

The substrates are incubated with sucrose and the glucansucrase at conditions that allow the enzyme to be active and to synthesize the maximum possible of the desired glucosides.

In particular, the concentrations and relative ratios of the sucrose, glucansucrase and substrate are chosen such that the enzymatic reaction may proceed to provide the desired product in optimum yield and selectivity.

In an embodiment, the reaction mixture comprises sucrose at a concentration from 50 to 400 g/l, more preferably 150 to 350 g/l, and most preferably 250 to 320 g/l.

In an embodiment, the temperature of the reaction mixture is maintained at 27 to 33° C., more preferably at 28 to 32° C., and most preferably at 29 to 31° C.

Optionally, the reaction mixture may further comprise calcium cations, e.g. in the form of calcium chloride or in the form of any other water soluble calcium salt, to improve the stability of the enzyme.

In an embodiment, the reaction mixture comprises calcium chloride in a concentration of from 0.4 to 4.0 g/l, more preferably of 0.7 to 3.5 g/l, and most preferably of 0.9 to 2.25 g/l.

In an embodiment, the epigallocatechin gallate and the epigallocatechin, respectively, is incubated at an initial concentration of from 5 to 50 mM, more preferably from 10 to 40 mM, and most preferably from 15 to 35 mM.

The methods of the present invention may be conducted with any suitable glucansucrase, for example with a dextransucrase or an amylosucrase.

In an embodiment, the glucansucrase is a dextransucrase.

It has been found that dextransucrases are able to efficiently and selectively glucosylate epigallocatechin gallate and epigallocatechin.

The concentration of the glucansucrase, preferably the dextransucrase, should be adjusted to the concentration of the substrate and/or the sucrose. In particular, the use of too little glucansucrase may lead to a reduced yield, whereas the use of too much glucansucrase may lead to a decrease in regioselectivity.

In an embodiment, the concentration of the glucansucrase in the reaction mixture is from 0.4 to 4.0 U/ml, more preferably 0.7 to 3.5 U/ml and even more preferably 0.9 to 2.25 U/ml.

Thanks to the reaction conditions of the present invention, the desired epigallocatechin gallate 4′-O-α-monoglucoside and epigallocatechin 4′-O-α-monoglucoside, respectively, are formed with a very high selectivity.

In an embodiment, the monoglucoside is formed in a ratio of at least 2:1 relative to the sum of the di- and tri-glucosides, more preferably of at least 3:1, even more preferably of at least 4:1, and most preferably of at least 5:1. Ideally, only the monoglucoside or essentially only the monoglucoside is formed. The selective formation of the monoglucoside is highly desirable, as this product is China compliant for cosmetic applications.

In an embodiment, the 4′-O-α-monoglucoside is formed with a regioselectivity of at least 75% on a molar basis compared to other monoglucosides, more preferably of at least 85%, even more preferably of at least 90%, and most preferably of at least 92%. It has been found that, thanks to this high regioselectivity, bioaccumulation in the skin can be improved.

Preferable, the monoglucoside is formed with high selectivity and in high stereoselectivity.

In an embodiment, the 4′-O-α-monoglucoside (i.e. epigallocatechin gallate 4′-O-α-monoglucoside or epigallocatechin 4′-O-α-monoglucoside, depending on the substrate) represents at least 60% of the total glucosides formed, more preferably at least 65%, even more preferably at least 70%, and most preferably at least 75%. In this context, the term “glucosides” include all glucosides formed, i.e. any mono-, di- and tri-glucosides and with the glucosylation occurring at any position.

The present invention further encompasses the epigallocatechin gallate 4′-O-α-monoglucoside and the epigallocatechin 4′-O-α-monoglucoside, respectively, formed by the methods of the present invention.

As has been explained above, these products are formed with a significantly higher selectivity than previously observed. Furthermore, they were also found to exhibit an improved skin penetration and bioaccumulation. The products of the present invention are also microbiome friendly.

In a further aspect, the present invention also provides a method for providing a radiant skin complexion and/or age-spot removal, wherein epigallocatechin gallate 4′-O-α-monoglucoside and/or epigallocatechin 4′-O-α-monoglucoside is topically applied to the skin.

The method is cosmetic, i.e. non-therapeutic.

Surprisingly, it was found that epigallocatechin gallate 4′-O-α-monoglucoside and epigallocatechin 4′-O-α-monoglucoside have a much higher bioavailability in the skin than the respective aglycones.

For example, it was found that epigallocatechin gallate 4′-O-α-monoglucoside is mainly accumulated in the skin at a depth of about 65 □m, in a quantity that is almost double that of the aglycone, epigallocatechin gallate. A glucoside mixture comprising mono-, di- and tri-glucoside was found to have a skin penetration depth of about 50 □m. Detailed results are presented in the example section below.

In several in tubo, in vitro, ex vivo and clinical studies, epigallocatechin gallate 4′-O-α-monoglucoside and epigallocatechin 4′-O-α-monoglucoside were further found to exhibit various activities rendering it interesting for cosmetic applications, including antioxidant, antiglycation and lightening activities.

For example, it was found that the epigallocatechin gallate 4′-O-α-monoglucoside of the invention significantly reduced the reactive oxygen species (ROS) by −66% using an in vitro model, demonstrating the strong antioxidant activity.

Furthermore, in tubo testing showed that the epigallocatechin gallate 4′-O-α-monoglucoside of the invention is a potent antiglycant active, as demonstrated by the IC50 value of 0.118 mM, which is better than the positive reference used in the test (aminoguanidine) and other well-known molecules, like Vitamin C.

The epigallocatechin gallate 4′-O-α-monoglucoside of the invention also has an excellent lightening activity: In an ex vivo test, the epigallocatechin gallate 4′-O-α-monoglucoside of the invention was topically applied to a human skin explant and compared to kojic acid as a positive control. It was found that epigallocatechin gallate 4′-O-α-monoglucoside provided a reduction of the melanin content by −34%, which is similar to that of kojic acid.

A clinical evaluation on female Asian volunteers revealed that the epigallocatechin gallate 4′-O-α-monoglucoside of the invention can improve the skin homogeneity and leads to a lighter skin tone and a reduction of pigmented spots, resulting in a more homogenous light skin tone. Indeed, a significant increase of skin homogeneity—up to 3.8 times better than the placebo after 56 days of twice daily application—was observed. In addition, a strong reduction of the melanin content in pigmented spots area of up to −21.7% in comparison to the placebo was also observed. Thus, in comparison to vitamin C at 2%, which is a common benchmark, the epigallocatechin gallate 4′-O-α-monoglucoside of the invention induced similar effect and was even performing better.

The epigallocatechin gallate 4′-O-α-monoglucoside of the invention was also found to significantly improve the skin firmness up to 21 times better than placebo after 56 days. Furthermore, a significant increase of the dermis density of up to 16.6% was observed.

In another clinical study with Caucasian volunteers having pigmented spots on their hands, it was demonstrated that the epigallocatechin gallate 4′-O-α-monoglucoside of the invention is able to significantly reduce the skin heterogeneity, with an improvement of skin tone and a reduction of pigmented skin. Indeed, a significant reduction of skin heterogeneity of up to −24% was observed, meaning that the epigallocatechin gallate 4′-O-α-monoglucoside was more efficient than vitamin C.

A detailed description of the biological tests is presented further down in the examples section.

Overall, these results demonstrated that the epigallocatechin gallate 4′-O-α-monoglucoside and the epigallocatechin 4′-O-α-monoglucoside of the invention are very well suited for skin care products providing a whitening effect, anti-age spots, skin complexion and lightening activity.

Consequently, the epigallocatechin gallate 4′-O-α-monoglucoside and epigallocatechin 4′-O-α-monoglucoside of the invention provide many benefits, rendering them interesting for cosmetic applications, such as anti-ageing, face care, and skin care products in general.

In an embodiment, the epigallocatechin gallate 4′-O-α-monoglucoside and the epigallocatechin 4′-O-α-monoglucoside, respectively, is formed by the method according to the present invention.

Preferably, the 4′-O-α-monoglucoside is present in a high proportion compared to other glucosides.

In an embodiment, the ratio of epigallocatechin gallate 4′-O-α-monoglucoside to other glucosides of epigallocatechin gallate is at least 2:1, more preferably at least 3:1, even more preferably at least 4:1, and most preferably at least 5:1.

In an embodiment, the ratio of epigallocatechin 4′-O-α-monoglucoside to other glucosides of epigallocatechin is at least 2:1, more preferably at least 3:1, even more preferably at least 4:1, and most preferably at least 5:1.

As mentioned above, the monoglucosides exhibit a particularly high bioavailability in the skin.

In an embodiment, the epigallocatechin gallate 4′-O-α-monoglucoside and the epigallocatechin 4′-O-α-monoglucoside, respectively, forms part of a cosmetic composition.

Cosmetic compositions typically comprise a cosmetically acceptable carrier and one or more cosmetic actives—in the present instance, the epigallocatechin gallate 4′-O-α-monoglucoside and/or the epigallocatechin 4′-O-α-monoglucoside.

Therefore, in an embodiment, a cosmetic composition comprising the epigallocatechin gallate 4′-O-α-monoglucoside and a cosmetically acceptable carrier is topically applied to the skin.

In an embodiment, a cosmetic composition comprising the epigallocatechin 4′-O-α-monoglucoside and a cosmetically acceptable carrier is topically applied to the skin.

The cosmetic composition comprises the epigallocatechin gallate 4′-O-α-monoglucoside and/or the epigallocatechin 4′-O-α-monoglucoside in an amount effective to provide the desired activity.

In an embodiment, the cosmetic composition comprises about 0.0001% to about 0.03% of the epigallocatechin gallate 4′-O-α-monoglucoside, more preferably about 0.001% to about 0.02% and most preferably about 0.005% to about 0.01%.

In an embodiment, the cosmetic composition comprises about 0.000075% to about 0.0225% of the epigallocatechin 4′-O-α-monoglucoside, more preferably about 0.00075% to about 0.015% and most preferably about 0.00375% to about 0.0075%.

The cosmetic composition used according to the invention may comprise one or more further cosmetic actives, in addition to the epigallocatechin gallate 4′-O-α-monoglucoside and/or the epigallocatechin 4′-O-α-monoglucoside.

For example, the cosmetic composition may comprise a skin lightening agent, a dark spot eraser, a skin tone agent, a hydrating agent, a moisturizer, a UV protection agent, an anti-ageing active, and/or a soothing agent.

In an embodiment, the cosmetic composition comprises one or more further cosmetic actives selected from the group consisting of lightening agents, dark spot erasers, skin tone agents, hydrating agents, moisturizers, UV protection actives, and/or anti-ageing actives.

Examples of lightening agents that may be used in the composition of the present invention include niacinamide, 12-hydroxystearic acid, resorcinol, phenylethyl resorcinol, 4-alkyl substituted resorcinol compounds, glutathione precursors, vitamin B6, vitamin C, vitamin A, caffeine, gallic acid and its derivatives, galardin, adapalene, aloe extract, sage extract, ginger extract, ammonium lactate, arbutin, azelaic acid, butyl hydroxy anisole, butyl hydroxy toluene, citrate esters, deoxyarbutin, 1,3-diphenyl propane derivatives, 2,5-dihydroxybenzoic acid and its derivatives, 2-(4-acetoxyphenyl)-1,3-dithiane, 2-(4-hydroxyphenyl)-1,3-dithiane, ellagic acid, glucopyranosyl-1-ascorbate, gluconic acid, glycolic acid, 4-Hydroxy-5-methyl-3[2H]-furanone, 4-hydroxyanisole and its derivatives, 4-hydroxybenzoic acid derivatives, hydroxycaprylic acid, inositol ascorbate, lactic acid, lemon extract, linoleic acid, magnesium ascorbyl phosphate, 5-octanoyl salicylic acid, salicylic acid, 3,4,5-trihydroxybenzyl derivatives, acetylglucosamine, pitera extract, symwhite, calcium pantothenate (Melano-block), seppiwhite, soybean extract (bowman birk inhibitor) and mixtures thereof. When 12-hydroxystearic acid is used in the composition, it is used as a skin lightening agent not as a fatty acid.

In addition or alternatively, the cosmetic composition of the present invention may further comprise plant extracts, for example a cranberry, hibiscus, guarana, acerola, ginger, licorice, pomegrentate, lotus, rhodiola and/or shizandra extract.

The cosmetic composition may also comprise one or more additives.

In an embodiment, the cosmetic composition further comprises a fatty acid.

Fatty acids when present in a composition along with a soap provide the so called vanishing effect, i.e. a composition, when applied on to the human skin, vanishes on the skin leaving behind no significant streaks of the composition.

Preferably, fatty acids that may be present in the composition are selected from fatty acids that have 10 to 30, more preferably 12 to 25, even more preferably 14 to 20, further more preferably 16 to 18 carbon atoms. Examples of fatty acids that may be used in the composition include pelargonic, lauric, myristic, palmitic, stearic, isostearic, oleic, linoleic, arachidic, behenic, erucic acid and mixtures thereof.

In an embodiment, the cosmetic composition further comprises soap.

Soap when present in combination with a fatty acid in the composition provides a vanishing effect.

Soap is preferably prepared by in-situ neutralization of fatty acid present in the composition. Thus, it is preferred that the soap has a carbon chain length that corresponds to the chain length of fatty acid in the composition. The soap is formed from the fatty acid through use of alkali metal hydroxides e.g. sodium hydroxide or potassium hydroxide. Of the two, potassium hydroxide is more preferred. Thus, the soap is preferably a potassium soap (potassium salt of fatty acid).

Preferably, the composition comprises from about 0.1 wt % to about 10 wt %, more preferably from about 1 wt % to about 8 wt %, more preferably from about 2 wt % to about 7 wt %, even more preferably from about 3 wt % to about 6 wt % soap.

In an embodiment, the cosmetic composition further comprises a surfactant, in particular a non-ionic surfactant having HLB value in the range 9 to 20, preferably 10 to 19, more preferably 12 to 18, even more preferably 13 to 17 and yet more preferably 15 to 17. HLB is calculated using the Griffin method wherein HLB=20×Mh/M wherein Mh is the molecular mass of the hydrophilic portion of the molecule and M is the molecular mass of the whole molecule, giving a result on an arbitrary scale of 0 to 20. Typical values for various surfactants are given below:
A value<10: Lipid soluble (water insoluble)
A value>10: Water soluble
A value from 4 to 8 indicates an anti-foaming agent
A value from 7 to 11 indicates a W/O (water in oil) emulsifier
A value from 12 to 16 indicates oil in water emulsifier
A value from 11 to 14 indicates a wetting agent
A value from 12 to 15 is typical of detergents
A value of 16 to 20 indicates a solubiliser or a hydrotrope

Preferably, the nonionic surfactant having HLB value in the range 9 to 20 is selected from fatty alcohol ethoxylates, alkyl phenol ethoxylates, polyoxyethylene sorbitan alkyl esters and mixtures thereof. Preferably, the nonionic surfactants are ones with at least 9 alkylene oxide groups preferably at least 9 ethylene oxide groups.

Preferably, the composition comprises from about 0.5 wt % to about 5 wt %, more preferably from about 1 wt % to about 4 wt %, even more preferably from about 2 wt % to about 3 wt % nonionic surfactant having HLB in the range 9 to 20.

In an embodiment, the cosmetic composition further comprises a polymer. The polymer acts as thickener in the composition and improves sensorial properties of the composition. The polymer is preferably selected from the following classes:
acrylate/R-methacrylate copolymer e.g. acrylates/steareth-20 methacrylate copolymer (commercially available as Aculyn™ 22) and acrylates/beheneth-25 methacrylate copolymer (commercially available as Aculyn™ 28);
acrylate/R-methacrylate crosspolymer e.g. acrylates/steareth-20 methacrylate crosspolymer (commercially available as Aculyn™ 88);
acrylates copolymer (commercially available as Aculyn™ 33);
acrylate/R-alkyl acrylate crosspolymer e.g. acrylates/C10-C30 alkyl acrylate crosspolymer (commercially available as Pemulen™ TR-2);
copolymer of ammonium acryloyldimethyltaurate with vinyl pyrrolidone (commercially available as Aristoflex® AVC);
copolymer of sodium acryloyldimethyltaurate with vinyl pyrrolidone (commercially available as Aristoflex® AVS); and
crosspolymer of acryloyldimethyltaurate with R-alkyl acrylate and methyacrylate e.g. ammonium acryloyldimethyltaurate/beheneth-25 methacrylate crosspolymer (commercially available as Aristoflex® HMB and Aristoflex® BLV).

Preferably, the composition comprises from about 0.1 wt % to about 5 wt %, more preferably from about 0.5 wt % to about 4.5 wt %, even more preferably from about 1 wt % to about 4 wt %, further more preferably from about 1.5 wt % to about 3.5 wt %, still more preferably from about 2 wt % to about 3 wt % polymer.

In an embodiment, the cosmetic composition further comprises an emollient.

Examples of emollients that may be used in the composition include stearyl alcohol, glyceryl monoricinoleate, mink oil, cetyl alcohol, isopropyl isostearate, isobutyl palmitate, isocetyl stearate, oleyl alcohol, isopropyl laurate, hexyl laurate, decyl oleate, octadecan-2-ol, isocetyl alcohol, eicosanyl alcohol, behenyl alcohol, cetyl palmitate, din-butyl sebacate, isopropyl myristate, isopropyl palmitate, isopropyl stearate, butyl stearate, polyethylene glycol, triethylene glycol, lanolin, cocoa butter, corn oil, cotton seed oil, olive oil, palm kernel oil, rape seed oil, safflower seed oil, evening primrose oil, soybean oil, sunflower seed oil, avocado oil, sesame seed oil, coconut oil, arachis oil, castor oil, acetylated lanolin alcohols, petroleum jelly, mineral oil, butyl myristate, isopropyl linoleate, lauryl lactate, myristyl lactate, decyl oleate, myristyl myristate and mixtures thereof.

In an embodiment, the cosmetic composition further comprises a solvent.

Examples of solvents that may be used in the composition include ethyl alcohol, isopropanol, acetone, ethylene glycol monoethyl ether, diethylene glycol monobutyl ether, diethylene glycol monoethyl ether and mixtures thereof.

In an embodiment, the cosmetic composition further comprises a powder.

Examples of powders that may be used in the composition include chalk, talc, fullers earth, kaolin, starch, gums, colloidal silica sodium polyacrylate, tetra alkyl and/or trialkyl aryl ammonium smectites, chemically modified magnesium aluminium silicate, organically modified montmorillonite clay, hydrated aluminium silicate, fumed silica, carboxyvinyl polymer, sodium carboxymethyl cellulose, ethylene glycol monostearate and mixtures thereof.

In an embodiment, the cosmetic composition further comprises a preservative to protect against the growth of potentially harmful microorganisms.

Examples of ingredients that may be used as preservatives in the composition include alkyl esters of para-hydroxybenzoic acid, hydantoin derivatives, propionate salts, and a variety of quaternary ammonium compounds. More preferably, ingredients that may be used as preservative in the composition are sodium benzoate, iodopropynyl butyl carbamate, methylisothiazolinone, iodopropynylbutylcarbamate, phenoxyethanol, methyl paraben, propyl paraben, imidazolidinyl urea, sodium dehydroacetate, ethylhexylglycerin, benzyl alcohol, alkane diols and mixtures thereof. The alkane diols that are suitable for use as preservative are C6-C12 alkanes that are vicinally substituted with hydroxy groups. Illustrative examples include 1,2-octane diol (caprylyl glycol), 2,3-octane diol, 1,2-nonane diol, 1,2-decane diol, 1,2-hexane diol, 3,4-octane diol, mixtures thereof or the like where caprylyl glycol is typically the most preferred.

When present in the composition, preservatives are added preferably in an amount of about 0.001 wt % to about 5 wt %, more preferably about 0.01 wt % to about 3 wt % and most preferably about 0.02 wt % to about 2 wt %.

The cosmetic composition may further comprise a range of other optional ingredients that include antioxidants, binders, biological additives, buffering agents, colorants, astringents, fragrance, opacifying agents, conditioners, exfoliating agents, pH adjusters, natural extracts, skin sensates, skin soothing agents, and skin healing agents.

The cosmetic composition is preferably formulated in the form of a compositions comprising water, such as a lotion, cream, emulsion, gel or mousse. More preferably, the composition is formulated in the form of cream or lotion and most preferably in the form of cream.

The present invention is further exemplified by means of the following non-limiting examples:

### EXAMPLE 1: SYNTHESIS OF EPIQALLOCATECHIN GALLATE 4′-O-α-MONOGLUCOSIDE (EGCG MONOG)

In a thermostated and agitated reactor, 2.40 kg (=2.401) of demineralized water were introduced. The temperature was adjusted at 30±1° C. Under mild agitation, 1.215 kg of sucrose (food grade) were introduced in fractions (about 6 fractions of 0.2 kg, each fraction being added once the previous one was completely dissolved). Subsequently, 4.0 g of Calcium chloride were introduced. An amount of 0.47 kg (=0.60 l) of 2-propanol (minimum purity: 98%) was introduced under mild agitation. 41.0 g of epigallocatechin gallate (EGCG; minimum purity: 95%) were introduced under mild agitation. Once completely solubilized, 0.162 kg of 0.50 M sodium acetate buffer at pH 5.41 were introduced into the reactor. At this point, the weight of the intermediary reaction mixture was 4.292 kg and its volume was 3.942 l.

0.840 kg (=0.840 l) of demineralized water was added and the pH was adjusted to 5.30 with 1 ml of 6N H₂SO₄. pH and temperature being at the nominal values of 5.3±0.1 and 30±1° C., 0.203 kg of enzyme solution (35.0 U/g; 1 U corresponds to the amount of enzyme that releases 1 μmol of fructose from sucrose per minute; in the conditions of the assay: sucrose 100 g/l, pH 5.20 with 20 mM sodium acetate buffer, calcium chloride 0.02 g/l). In total, the mass of the reaction mixture was 5.247 kg and the volume 4.897 l. The EGCG concentration was 7.8 g/kg (17.0 mmol/kg) or 8.4 g/l (18.3 mM). The dextransucrase concentration was 0.76 U/g or 0.81 U/ml.

The reaction was carried out during 24 hours. The enzymatic reaction was stopped by adjusting the pH of the reaction mixture to a value of 2.0 using concentrated sulfuric acid. EGCG-related substances were separated from sugars, enzyme and salts by adsorption using a hydrophobic resin (Diaion HP20 from Mitsubishi Chemical Industries, Japan), followed by elution with an ethanol/water mixture (specific gravity at 20° C.: 0.862). The ethanol/water mixture used for the elution of the EGCG-related substances was removed by vacuum evaporation. EGCG-glucosides were separated from EGCG using liquid-liquid extraction with ethyl acetate (the concentrations of substances and relative volume of ethyl acetate were adjusted in order to maximize the purification yield). Such methods are known to the skilled person and described elsewhere, in particular in WO 2007/144368 (pages 46-47).

The final composition of the purified preparation was as follows:
monoglucosides: 41.7317 mmol/kg (corresponding to 87.77% of the glucosides)
diglucosides: 5.813 mmol/kg (corresponding to 12.23% of the glucosides)
triglucosides: not observed
residual EGCG: 0.4466 mmol/kg

Epigallocatechin gallate 4′-O monoglucoside was the main constituent, representing 79.10% of the monoglucosides.

This purified preparation containing about 69% of epigallocatechin gallate 4′-O monoglucoside was used in the tests described below.

### EXAMPLE 2: SYNTHESIS OF EPIGALLOCATECHIN 4′-O-α-MONOGLUCOSIDE

Epigallocatechin 4′-O-α-monoglucoside was prepared under the same reaction conditions as described in Example 1, replacing epigallocatechin gallate by epigallocatechin as the substrate.

### EXAMPLE 3: STATISTICAL ANALYSIS

For the in vitro and ex vivo studies described in Examples 4 to 8, all results are presented as mean±standard error of mean (SEM) of three independent triplicates. The in vivo data of Examples 11 to 14 are typically expressed in percent of variation compared to D0.

A Shapiro Wilk test was used to verify whether the raw data followed the Gaussian Law. In case of Normally-distributed data, the mean values were compared using either an unpaired t test (s 2 groups) or One-way ANOVA followed by post-hoc test (≥2 groups). In case of non-Normally-distributed data, a Kruskal-Wallis test followed by a Mann-Whitney U test was used for unpaired data.

For all in vivo studies, a Shapiro Wilk test was used to verify whether the raw data followed the Gaussian Law. In case of Normally-distributed data, the mean values were compared using either an unpaired or paired Student's t-test. In case of non-Normally-distributed data, a Wilcoxon (paired) or Kruskal-Wallis test followed by a Mann-Whitney U (unpaired) test were used for paired data or unpaired data, respectively. Concerning the analysis of the self-assessment questionnaire and daily log results, a Chi-square test was done (dichotomous analysis which consists in comparing the number of associated answers).

In all cases, results were considered as significant with p<0.1 with #, p<0.05 with *, p<0.01 with ** and p<0.001 with ***.

### EXAMPLE 4: ANTIOXIDANT ACTIVITY (IN VITRO)

### Cell Culture and Treatment

A 96 wells-plate was coated with a collagen I solution at 50 μg/ml. Normal Human Epidermal Keratinocytes (NHEKs) were seeded at 20′000 cells per well with four replicates. Cells were incubated for 24 hours in complete medium (Epilife, Gibco) supplemented with Human keratinocyte growth supplements (HKGS) factor (Gibco) and 1% antibiotics (Sigma) at 37° C. with 5% CO₂.

At the end of the incubation time, the cells were treated with the purified preparation from Example 1, containing epigallocatechin gallate 4′-O-α-monoglucoside, at 0.1% (v/v) or with Resveratrol at 200 μM (used as positive control for the assay), both in 2′,7′-dichlorodihydrofluorescein diacetate, for 24 hours at 37° C. with 5% CO₂. After 24 hours, cells were incubated with the DCF H-DA probe (culture medium) at 50 μM for 30 to 40 minutes at 37° C. with 5% CO₂ and stressed with the tert-butylperoxide (TBP) at 5 mM. The evolution of the fluorescence was followed for 1 hour.

### Results

Resveratrol at 200 μM significantly reduced the ROS production by 61% and validated the experiment.

Epigallocatechin gallate 4′-O-α-monoglucoside at 0.1% significantly reduced the ROS production by 66%, confirming an antioxidant activity on keratinocytes.

| | | | Resveratrol | ECGC |
|---|---|---|---|---|
| | Untreat- | | at | monoglucoside |
| | ed | TBP | 200 μM | at 0.1% |
| Mean (% of untreated) | 27.5 | 100 | 39 | 33.75 |
| SEM | 1.32 | 7.31 | 6.17 | 4.6 |

### EXAMPLE 5: ANTI-GLYCATION ACTIVITY (IN VITRO)

The anti-glycation effect was evaluated using an in tubo assay to measure the spontaneous formation of Advanced Glycation End-products (AGEs).

### Treatment

This assay was conducted on a 96-wells microplate. 40 μl of the purified preparation from Example 1, containing epigallocatechin gallate 4′-O-α-monoglucoside at 0.1% (v/v), were mixed with 50 μl of BSA and 10 μl of ribose. The extracts were diluted in sodium phosphate buffer (0.1 M, pH 7.4). Several dilutions were tested. Each diluted extract was run in triplicate.

A standard range of aminoguanidine was used as inhibitor reference (positive control). A mixture of the sodium phosphate buffer and BSA was used to validate the experiment and a mixture of sodium phosphate buffer, BSA and ribose was used as the positive control.

Two blanks were carried out for the extract:
with phosphate buffer, BSA and extract
with phosphate buffer, ribose and extract

The microplate was agitated and incubated for 17 h at 37° C. The fluorescence reading was performed with [symbol] excitation 340 nm and [symbol] emission 420 nm.

The anti-glycation activity of the extract was calculated as follows:

$$\% Inhibition of AGEs formation = \frac{1 - ( Fl ( extract ) - Fl ( blank extract ) )}{Fl ( positive control ) - Fl ( negative control )} * 100$$

FI: Fluorescence of tested extract; blank extract (ribose or BSA); positive control; negative control.

Based on the above measurements and calculation, the IC₅₀, i.e. the concentration of the extract which inhibits AGEs formation by half, was calculated.

### Results

The results are consolidated in the following table:

| | Initial | | Tested | | Inhibition | IC₅₀ |
|---|---|---|---|---|---|---|
| Extract | concentration | | concentrations | | AGE's | in well |
| EGCG | 10.9 | mM | 10.9 | mM | 97.36% | 0.128 mM |
| aglycone | | | 2.180 | mM | 93.56% | |
| | | | 0.436 | mM | 59.84% | |
| | | | 0.109 | mM | 31.95% | |
| ECGC | 47.99 | mM | 8 | mM | 109.26% | 0.133 mM |
| monoglucoside | | | 1 | mM | 81.54% | |
| at 0.1% | | | 0.4 | mM | 53.22% | |
| | | | 0.08 | mM | 31.01% | |

The IC₅₀ of the positive control (aminoguanidine) was determined as 34.7 □g/ml (0.326 mM).

### EXAMPLE 6: EVALUATION OF THE SKIN PENETRATION BY RAMAN SPECTROSCOPY (EX VIVO)

### Skin Explants Culture and Treatment

Skin explants from a Caucasian volunteer were treated with epigallocatechin gallate, i.e. the aglycone, with a glucoside mixture comprising epigallocatechin gallate mono-, di- and tri-glucoside, or with the purified preparation from Example 1, containing epigallocatechin gallate 4′-O-α-monoglucoside, at 2% for 8 hours at 37° C. with 5% CO₂. At the end of the incubation period, the skin explants were frozen in Optimal Cutting Temperature (OCT; VWR) at −80° C. and then cut longitudinally using a cryotome with a thickness of 20 μm. For each explant, 3 tissue sections were selected and deposited on a CaF₂ support for Raman imaging analysis. The processing of corrected data maps was performed by using a software based on least squares fitting method that operates in the Matlab environment. This method involves mathematical modeling of reference spectra in the overall spectral image to determine the contribution and distribution of these spectra within the image. The reference spectra were the modelled spectra of the following products: Aglycone, glucoside mixture, 4′-O-α-monoglucoside and untreated skin control.

### Results

It was found that, after 8 hours of topical application, the skin penetration of epigallocatechin gallate 4′-O-α-monoglucoside was significantly higher than that of the aglycone (+90%) and that of the glucoside mixture (+20%).

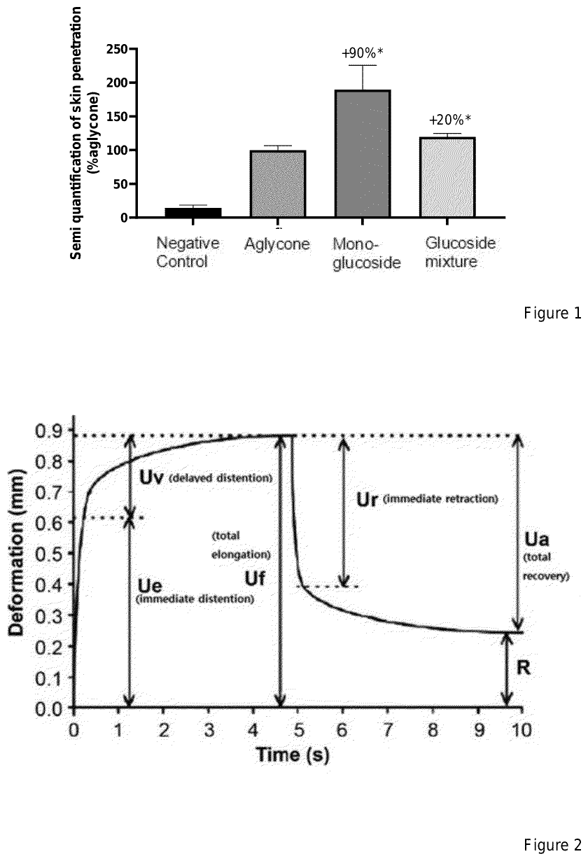

FIG. 1 shows a semi-quantitative representation of the results.

Thus, the epigallocatechin gallate 4′-O-α-monoglucoside of the invention has a better skin bioavailability than the aglycone and the glucoside mixture.

### EXAMPLE 7: EVALUATION OF WHITENING ACTIVITY (EX VIVO) USING FONTANA-MASSON STAINING FOR MELANIN QUANTIFICATION

### Skin Explants Culture and Treatment

Fresh human skin explants were obtained from skin phototype III and VI donors, aged of 46±25 years. The skin explants were topically treated with epigallocatechin gallate 4′-O-α-monoglucoside (purified preparation from Example 1) at 0.4% (v/v) or with vitamin at C 2% (v/v), both in water, for 7 days and incubated at 37° C., 5% CO₂. Kojic acid (Sigma-Aldrich) at 2% was topically applied and incubated under the same conditions, serving as positive control. The untreated condition was treated with the vehicle (distilled water). The medium was renewed every other day. After the end of culture, the skin explants were sampled and put in formalin before being dehydrated and embedded in paraffin for Fontana-Masson staining.

### Fontana-Masson Staining and Melanin Quantification by Image Analysis

Skin explants fixed in formalin were dehydrated and embedded in paraffin. Sections of 4 μm thickness were dewaxed and then stained by Fontana-Masson silver method for melanin.

Images were collected using an Axio Observer Inverted fluorescence microscope (Zeiss) in bright field mode.

Melanin was quantified using two open source optical imaging software programs. Photomicrographs (jpeg format) of Fontana-Masson tissue sections were opened in GIMP-GNU Image Manipulation Program. The brown-black color signals corresponding to melanin grains on stained sections were selected, copied and pasted into a new image and saved as a jpeg file; this jpeg file consists solely of black/brown (melanin grains) on a white background. This image was subsequently opened using the ImageJ program. The image was then inverted and the mean intensity was obtained.

### Results

Kojic acid at 2% and vitamin C at 2% significantly reduced the melanin content by 34% and 28%, respectively, compared to the untreated condition and validated the experiment.

Epigallocatechin gallate 4′-O-α-monoglucoside at 0.4% significantly reduced the melanin content by 34% compared to the untreated condition.

### EXAMPLE 8: TRANSCRIPTOMIC ANALYSIS

To evaluate the impact of preparation of the present invention on melanogenesis, a co-culture study with keratinocytes and melanocytes was conducted.

### Cell Culture and Treatment

The cell culture was realized with primary cells isolated from biopsies. Cells were co-cultured with a ratio of 10 keratinocytes to 1 melanocyte. Both cell types were derived from a 7-year-old Caucasian male donor.

The experiment was carried out under the following conditions:
NHEK-NHEM co-culture untreated
NHEK-NHEM co-culture+a diluted version of the purified preparation from Example 1, containing epigallocatechin gallate 4′-O-α-monoglucoside at 0.01% (v/v) (abbreviated as “EGCG MONOG 0.01%”)

The product EGCG MONOG 0.01% was filtered and diluted in Keratinocyte Growth Medium 2 (supplied by Promocell).

Cells were seeded in 6-well plates in the presence of the Keratinocyte Growth Medium 2 for 24 h. After seeding, a treatment in the presence of EGCG MONOG 0.01% was applied for 24 h. Untreated wells were used as a basal reference in order to compare the impact of EGCG MONOG and gene expression after 24 h of incubation.

After treatment, the RNA from the cells were extracted using TRIzol™ (available from Sigma) and reverse-transcribed into cDNA. The cDNA was mixed with different primers of interest representative of the melanogenesis pathway (PrimePCR custom plate, pigmentation, Biorad). The expression of the mRNA was measured by semi-quantitative PCR on a Bio-Rad CFX96Real Time PCR System (available from BioRAD). The expression level of the mRNA was calculated and normalized with reference gene (B2M).

The values were reported relative to the untreated control to express an activating or inhibitory effect.

### Results

After 24 h of treatment with EGCG MONOG 0.01%, the RT-qPCR revealed that EGCG MONOG 0.01% was able to significantly reduce the expression of genes involved in the regulation of melanogenesis (KIT −93%, EDNRB −68%, MC1R −96%, SOX10 −60%, MITF −108%). EGCG MONOG 0.01% was also able to significantly reduce the synthesis of melanin (TYR −58% and TYRP1-95%), the melanosome biogenesis (AP3B1 −59%, GPR 143-365%, PMEL −212%, DTNBP1 −59% and LYST −48%) and the melanine uptake by keratinocytes (F2RL1 −49%) in comparison to the untreated co-culture.

### EXAMPLE 9: CONSUMPTION OF EGCG MONOG BY BACILLUS SP

As the active of the present invention is glycosylated, it was hypothesized that it could be used by bacteria as a substrate. To test this hypothesis, Bacillus sp. was cultivated in the presence of EGCG MONOG at 1%, and its consumption was tracked by HPLC during 48 hours.

### Materials and Methods

Bacillus sp. was isolated from normal and healthy skin (GAB04-01) and was used for this study. Bacterial strains are stored at-80° C. in the presence of 20% w/v glycerol.

In preparation of the study, a selected sample was thawed and an aliquot (a cryotube of bacteria stored at −80° C.) was spread on a Tryptic Soy Agar plate before incubation at 30° C. A single colony was used to inoculate 10 ml of Tryptic Soy Broth (TSB) in a stoppered plastic tube and incubated at 37° C. overnight under agitation. The optical density at 600 nm was measured after a 10-fold dilution with TSB medium (Biophotometer D30). An aliquot of the sample was first diluted with fresh culture medium (Difco® Sporulation Medium (DSM) 0.5) to obtain an optical density of 1.216.

1 ml of this suspension was used to inoculate 40 ml of fresh culture medium (DSM 0.5). 24 hours after starting the culture, the culture medium was changed by adding 1% of the purified preparation from Example 1, containing epigallocatechin gallate 4′-O-α-monoglucoside at 0.1% (v/v), or water (negative control) to the medium (i.e. the samples were treated with 39.6 ml of DSM 0.5 and either 0.4 ml of EGCG MONOG or 0.4 ml of water). The optical density of the reaction medium just after inoculation was 0.030±0.003.

The fate of EGCG MONOG in the presence of Bacillus sp. was followed using HPLC-UV-MS with the following parameters:
Elution profile: 1.00 ml/min
Column: X-Bridge C18
Injection: 10 μl
Temperature: 40° C.
Spectrophotometer: 270 nm

### Results

After only 24 hours, the quantity of EGCG MONOG was reduced by 0.307 mM. At the same time, EGCG aglycone or any other EGCG derivatives (such as EGCG diglycosides) could not be detected, proving that the reduction of EGCG MONOG was linked to its consumption by bacteria and not its degradation. Detailed results are shown in the following table:

| | EGCG | EGCG | EGCG |
|---|---|---|---|
| Time | Monoglucoside | Diglycoside | Aglycone |
| 24 h | 0.751 nM | 0.117 nM | 0.009 nM |
| 27 h | 0.678 nM | 0.122 nM | 0.035 nM |
| 30 h | 0.614 nM | 0.118 nM | 0.038 nM |
| 48 h | 0.443 nM | 0.088 nM | 0.013 nM |
| 54 h | 0.434 nM | 0.099 nM | 0.006 nM |
| 72 h | 0.288 nM | 0.080 nM | 0.002 nM |
| Variation | −0.307 nM | −0.029 nM | −0.005 nM |
| over 48 h | | | |

### EXAMPLE 10: GROWTH PROMOTION OF LACTOBACILLUS ACIDOPHILUS

In order to further investigate the effect of the active of the present invention on bacteria, the growth of Lactobacillus acidophilus in MRS medium supplemented with different concentrations of EGCG MONOG was evaluated for 48 hours.

### Materials and Methods

L. acidophilus (DSM 20079) was cultivated at 37° C. under anaerobic conditions on De Man, Rogosa Sharpe (MRS) agar. After bacterial amplification in culture medium, for each bacterial strain, a bacterial suspension was prepared using the assay medium (MRS broth 50% in PBS) and adjusted to an optical density at 600 nm (OD600 nm) of 0.2. The bacterial suspension was then transferred to 96-well plates, some of which contained the test compounds from 0.015% to 1%. The strain cultivated with powdered milk at 0.01% served as a positive control. Optical density of bacterial suspension was measured using a wavelength of 600 nm and showed a value of 0.1.

Bacterial cultures were incubated at 37° C. under agitation (˜280 rpm) and under anaerobic condition. OD600 nm were read in kinetic for 48 hours using a microplate spectrophotometer (EPOCH2, BioTek Instruments) to analyze the bacterial growth.

All experimental conditions were performed in triplicate.

### Results

Milk powder at 0.01% significantly increased the growth of the bacteria and validated the experiment.

The active of the present invention was able to significantly increased the growth of Lactobacillus acidophilus with a dose-dependent effect by 6%, 9% and 27%, respectively, at concentrations of 0.13%, 0.4% and 1%, respectively, in comparison to the culture without EGCG MONOG.

### EXAMPLE 11: ANALYSIS OF METABOLITES RELEASED BY LACTOBACILLUS ACIDOPHILUS

As the active of the present invention was able to stimulate the growth of L. acidophilus, it was decided to analyze the metabolites released by the bacteria in the culture medium by high-field NMR.

### Materials and Methods

The supernatants of the cultures previously described in Example 10, both of the untreated strain culture and the culture of L. acidophilus treated with 1% EGCG MONOG were kept and frozen at −80° C. before analysis using high-field NMR. For each of these two conditions, three samples were analyzed to assess the tests reproducibility (n=3).

For each sample, 400 μl of supernatant were taken and 100 μl of D₂O/TSP (3-(trimethylsilyl)propionic-2,2,3,3-d4 acid sodium salt, internal reference) were added at a concentration of 1 g/l. The samples were analyzed by high field ¹H NMR on a Bruker ASCEND 500 spectrometer operating at a proton frequency of 500 MHz using a 5 mm cryosonde probe. NMR spectra were calibrated to TSP-d4 at 0 ppm using Topspin software version 4.3.0 (Bruker Biospin, Germany). Quantification of metabolites was performed by utilizing the internal standard (TSP-d4) added at a known concentration. Compounds were quantified by the relative ratio of the intensities of their peak integrals and those of the internal standard.

### Results

It was found that the addition of the active of the present invention at 1% changed the carbon source of the strain for its metabolism. Untreated bacteria mainly used sugars as carbon source, whereas treatment with EGCG MONOG lead to a decreased consumption of sugars by 17% in favor of polysorbate consumption, which significantly increased by 51%.

In addition, following culture with the active of the present invention, L. acidophilus released significantly more Trigonelline by up to 65%.

### EXAMPLE 12: COMPOSITIONS USED IN CLINICAL STUDIES

A clinical study using a facial cream containing 0.4% of the purified preparation from Example 1, which contains epigallocatechin gallate 4′-O-α-monoglucoside, or 2% of ascorbic acid (vitamin C) versus a placebo cream was performed in order to evaluate the efficacy on volunteers with dull skin, large senescence spots (superior 3 mm) on the face and with shallow wrinkles. For this study, volunteers applied the cream twice a day for 56 days.

For the clinical studies described in Examples 14 to 17, the following compositions were used: INCI formulae
Placebo: AQUA/WATER, CETHYL ALCOHOL, GLYCERYL STEARATE, PEG-75 STEARATE, CETETH-20, STEARETH-20, ISODECYL NEOPENTANOATE, PHENOXYETHANOL, 1-2-HEXANEDIOL, CAPRYLYL GLYCOL, DIMETHICONE, FRAGRANCE
Active: AQUA/WATER, CETHYL ALCOHOL, GLYCERYL STEARATE, PEG-75 STEARATE, CETETH-20, STEARETH-20, ISODECYL NEOPENTANOATE, EPIGALLOCATECHIN GALLATE MONOGLUCOSIDE, PHENOXYETHANOL, 1-2-HEXANEDIOL, CAPRYLYL GLYCOL, DIMETHICONE, FRAGRANCE
Vitamin C: AQUA/WATER, CETHYL ALCOHOL, GLYCERYL STEARATE, PEG-75 STEARATE, CETETH-20, STEARETH-20, ISODECYL NEOPENTANOATE, ASCORBIC ACID, PHENOXYETHANOL, 1-2-HEXANEDIOL, CAPRYLYL GLYCOL, DIMETHICONE, FRAGRANCE

### Compositions

| | Placebo | Vitamin C | Active |
|---|---|---|---|
| AQUA/WATER | 89.3% | 87.3% | 88.9% |
| CETHYL ALCOHOL, GLYCERYL | 5.0% | 5.0% | 5.0% |
| STEARATE, PEG-75 STEARATE, | | | |
| CETETH-20, STEARETH-20 | | | |
| ISODECYL NEOPENTANOATE | 4.5% | 4.5% | 4.5% |
| EPIGALLOCATECHIN GALLATE | — | — | 0.4% |
| MONOGLUCOSIDE* | | | |
| ASCORBIC ACID | — | 2% | — |
| PHENOXYETHANOL | 0.5% | 0.5% | 0.5% |
| 1-2-HEXANEDIOL, CAPRYLYL | 0.3% | 0.3% | 0.3% |
| GLYCOL | | | |
| DIMETHICONE | 0.3% | 0.3% | 0.3% |
| FRAGRANCE | 0.1% | 0.1% | 0.1% |

*The purified preparation from Example 1 contains 32.6933 mmol/kg = 20.2869 g/kg of epigallocatechin gallate 4′-O-α-monoglucoside, resulting in a final concentration in the tested composition of 0.0811 g/l = 0.0081%.

### EXAMPLE 13: PANEL USED IN CLINICAL STUDIES

The clinical studies described in Examples 14 to 16 were conducted as follows:

A single-center study with 38 Asian volunteers with dull skin and large senescence spots (superior to 3 mm) on the face and presenting shallow wrinkles was carried out. Volunteers were divided into two groups as follows:
Group 1: 20 Asian women testing the active cream in comparison to the placebo cream with an average age of 54±5 years.
Group 2: 18 Asian women testing the vitamin C cream in comparison to the placebo cream with an average age of 53±8 years.

The study was conducted according to the standard operating procedure of Bio EC and in compliance with the regulations established in “Gula para investigaciones con seres humanos” (Guidelines for Research on Human Beings) and the guidelines of the Scientific Committee on Consumer Safety (SCCS).

Volunteers applied a cream containing 0.4% of active, 2% of ascorbic acid (vitamin C), respectively, on one hemiface and a placebo cream on the other hemiface every other day for 56 days.

During this study, a skin color and spots analysis was conducted using Colorface®, biomechanical properties were tested using Cutometery®, and the dermis thickness was determined using a DUB SkinScanner 75® probe (max. depth of 3.28 mm).

### EXAMPLE 14: SKIN BIOMECHANICAL PROPERTIES MEASUREMENT BY CUTOMETER® METHOD

The analysis of the mechanical properties of the skin enables to assess the functional state of the following tissue structures:
The elastic structures, such as elastic fibers, curvature of the connective bundles, wrinkles of the stratum corneum; and
the viscous-behaving structures, such as interstitial fluids, internal adherences.

The study was performed using a Cutometer® MPA 580 (Courage & Khazaka). The measuring principle is based on the suction method: Negative pressure is created in the device and the skin is drawn into a cylindrical aperture (2 mm in diameter) of the probe. Inside the probe, the penetration depth is determined by an optical measuring system. Each suction phase is followed by a relaxing phase.

In the present study, the following program was used:
Length of the cycle: 4 seconds
Suction: 2 seconds
Relaxation: 2 seconds
Negative pressure: 450 millibars
Diameter of the chamber: 2 mm
Area measured: crow's feet

The resistance of the skin to be sucked up by the negative pressure and its ability to return to its original position are displayed as curves at the end of each measurement. From these curves, the parameters can be calculated.
During the suction phase, the deformation of the skin by the negative pressure measures first the elastic resistance and then the viscous component, which taken together represent skin firmness.
During the relaxation phase, the immediate recovery of the skin measures sheer cutaneous elasticity, whereas the delayed return of the skin to its initial position measures the viscoelastic component.

FIG. 2 shows an example of a curve measured on elastic skin. The following parameters are indicated:

| Parameter | Parameter mean |
|---|---|
| R0 | Uf (Total elongation) |
| R1 | Uf − Ua (Return to original skin) |
| R2 | Ua/Uf (Gross elasticity) |
| R3 | Last maximal amplitude |
| R4 | Last minimal amplitude |
| R5 | Ur/Ue (Net elasticity) |
| R6 | Uv/Ue (Viscoelasticity) |
| R7 | Ur/Uf (Skin firmness) |
| R8 | Ua (Total recovery) |

This study focused on the R0 or Uf parameter, which represents the amplitude of the skin during the suction phase. At equal pressure, the more flexible the skin, the greater the amplitude. Thus R0 (or UF) will evaluate the viscoelastic dispensability, or in other words, the firmness of the skin.

These parameters were measured on D0, D28, D56.

### Results

After 28 and 56 days of application, a decrease of the R0 parameter was observed, which indicates an increase of firmness with the epigallocatechin gallate 4′-O-α-monoglucoside (˜8.7% at D28 and −8.2% at D56) in comparison to D0. The results for epigallocatechin gallate 4′-O-α-monoglucoside and ascorbic acid, respectively, are shown in the following two tables:

| | Active | | | Placebo | | | Unpaired |
|---|---|---|---|---|---|---|---|
| Firmness | Mean +/− | Average | Paired | Mean +/− | Average | Paired | t test |
| effect | SD (arbi- | variation | t test | SD (arbi- | variation | t test | versus |
| (R0) | trary unit) | (%) vs D 0 | versus D 0 | trary unit) | (%) vs D 0 | versus D 0 | placebo |
| D 0 | 0.281 ± 0.046 | | | 0.252 ± 0.044 | | | |
| D 28 | 0.257 ± 0.041 | −8.7% | ** p < 0.01 | 0.250 ± 0.047 | −0.9% | ns | * p < 0.05 |
| D 56 | 0.256 ± 0.038 | −8.2% | ** p < 0.01 | 0.250 ± 0.046 | −0.4% | ns | * p < 0.05 |

| | | | | | | | Mann |
|---|---|---|---|---|---|---|---|
| | Ascorbic acid | | | Placebo | | | Whitney |
| Firmness | Mean +/− | Average | Wilcoxon | Mean +/− | Average | Wilcoxon | test |
| effect | SD (arbi- | variation | test | SD (arbi- | variation | test | versus |
| (R0) | trary unit) | (%) vs D 0 | versus D 0 | trary unit) | (%) vs D 0 | versus D 0 | placebo |
| D 0 | 0.283 ± 0.036 | | | 0.256 ± 0.042 | | | |
| D 28 | 0.263 ± 0.032 | −6.9% | ** p < 0.01 | 0.255 ± 0.041 | −0.3% | ns | * p < 0.05 |
| D 56 | 0.264 ± 0.046 | −6.5% | * p < 0.05 | 0.251 ± 0.041 | −1.9% | ns | ns |

These measurements confirmed a significant improvement of the skin firmness by up to 21 fold after application of the cream containing epigallocatechin gallate 4′-O-α-monoglucoside relative to the placebo cream.

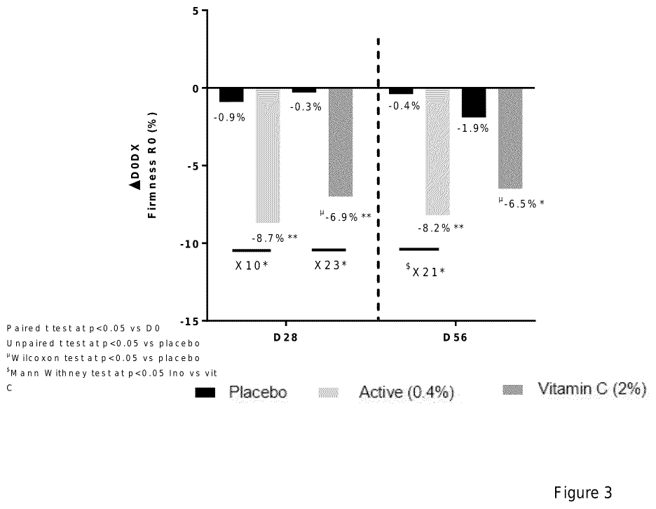

As can be seen in FIG. 3, the effect of epigallocatechin gallate 4′-O-α-monoglucoside was also significantly better than ascorbic after D28 and D56 days.

### EXAMPLE 15: SKIN COLOR ANALYSIS USING COLORFACE®

### Method

Skin color measurements were performed based on photos taken with the Colorface® acquisition system. Colorface® is a 2D acquisition system for taking standardized and multimodal photos of the entire face. The device is equipped with a 24M pixel sensor and also has a specific UV lamp. The acquisition methods are: UltraViolet, photo without filter, cross-polarized photo, parallel-polarized photo, standard 45° photo, and standard 600 photo.

An analysis of the radiance of the complexion, pigment spots, color (L*,a*,b* parameters) and melanin on the spots from photos using image analysis software was performed. 2D photos of the face were taken on D0, D28 and D56.

### Results Homogeneity

After 28 and 56 days of application of the active cream, a significant increase of the mean pigment homogeneity of +1.2% and +1.5%, respectively, was observed with the active in comparison to D0.

With the placebo, a significant increase of 0.4% was shown relative to D0 after 28 and 56 days.

Thus, the effect on pigment homogeneity was 3.0 times more pronounced after 28 days and 3.8 times more after 56 days for the product containing epigallocatechin gallate 4′-O-α-monoglucoside relative to the placebo.

The results for the active were significant in comparison with both D0 and the placebo as can be seen from the following table:

| | Active | | | Placebo | | | Unpaired |
|---|---|---|---|---|---|---|---|
| Pigment | Mean +/− | Average | Paired | Mean +/− | Average | Paired | t test |
| homoge- | SD (arbi- | variation | t test | SD (arbi- | variation | t test | versus |
| neity | trary unit) | (%) vs D 0 | versus D 0 | trary unit) | (%) vs D 0 | versus D 0 | placebo |
| D 0 | 92.2 ± 2.0 | | | 93.2 ± 1.5 | | | |
| D 28 | 93.3 ± 2.1 | 1.2% | *** | 93.6 ± 1.6 | 0.4% | # p < 0.1 | * p < 0.5 |
| D 56 | 93.6 ± 2.0 | 1.5% | *** | 93.6 ± 2.0 | 0.4% | ns | *** p < 0.001 |

For comparison, the results with ascorbic acid are shown in the following table:

| | Active | | | Placebo | | | Unpaired |
|---|---|---|---|---|---|---|---|
| Pigment | Mean +/− | Average | Paired | Mean +/− | Average | Paired | t test |
| homoge- | SD (arbi- | variation | t test | SD (arbi- | variation | t test | versus |
| neity | trary unit) | (%) vs D 0 | versus D 0 | trary unit) | (%) vs D 0 | versus D 0 | placebo |
| D 0 | 92.4 ± 1.6 | | | 93.4 ± 2.5 | | | |
| D 28 | 93.4 ± 1.6 | 1.1% | *** p < 0.001 | 94.1 ± 2.5 | 0.7% | ** p < 0.01 | # p < 0.01 |
| D 56 | 93.6 ± 2.0 | 1.4% | *** p < 0.001 | 94.2 ± 2.6 | 0.8% | *** p < 0.001 | * p < 0.05 |

Thus, vitamin C is only 1.6 and 1.8 fold, respectively, better than the placebo at days 28 and 56.

### Results Radiance

As can be seen from the results in the following table, a clear increase of skin radiance was observed after 28 and 56 days of application of the active cream containing 0.4% of epigallocatechin gallate 4′-O-α-monoglucoside:

| | Active | | | Placebo | | | Unpaired |
|---|---|---|---|---|---|---|---|
| | Mean +/− | Average | Paired | Mean +/− | Average | Paired | t test |
| | SD (arbi- | variation | t test | SD (arbi- | variation | t test | versus |
| Radiance | trary unit) | (%) vs D 0 | versus D 0 | trary unit) | (%) vs D 0 | versus D 0 | placebo |
| D 0 | 72.3 ± 2.2 | | | 71.7 ± 1.7 | | | |
| D 28 | 72.9 ± 2.6 | 0.9% | ** p < 0.01 | 71.9 ± 2.4 | 0.2% | ns | # p < 0.1 |
| D 56 | 73.5 ± 2.5 | 1.3% | ** p < 0.01 | 72.3 ± 2.0 | 0.5% | ns | # p < 0.1 |

Compared to the placebo, a significant increase by 4.5 and 2.6 fold, respectively, was observed after 28 and 56 days.

For comparison, the results with ascorbic acid are shown in the following table:

| | | | | | | | Mann |
|---|---|---|---|---|---|---|---|
| | Ascorbic acid | | | Placebo | | | Whitney |
| | Mean +/− | Average | Wilcoxon | Mean +/− | Average | Wilcoxon | test |
| | SD (arbi- | variation | test | SD (arbi- | variation | test | versus |
| Radiance | trary unit) | (%) vs D 0 | versus D 0 | trary unit) | (%) vs D 0 | versus D 0 | placebo |
| D 0 | 72.3 ± 1.6 | | | 71.4 ± 2.2 | | | |
| D 28 | 72.7 ± 1.9 | 0.7% | * p < 0.05 | 72.1 ± 2.3 | 1.0% | *** p < 0.01 | ns |
| D 56 | 73.3 ± 2.3 | 1.4% | *** p < 0.001 | 72.1 ± 2.4 | 0.9% | *** p < 0.001 | ns |

### Results Pigment Defect Reduction

As can be seen from the results in the following table, a clear decrease of pigment defect was observed after 28 and 56 days of application of the active cream containing 0.4% of epigallocatechin gallate 4′-O-α-monoglucoside:

| | Active | | | Placebo | | | Unpaired |
|---|---|---|---|---|---|---|---|
| | Mean +/− | Average | Paired | Mean +/− | Average | Paired | t test |
| Pigment | SD (arbi- | volume | t test | SD (arbi- | volume | t test | versus |
| defect | trary unit) | (mm³) vs D 0 | versus D 0 | trary unit) | (mm³) vs D 0 | versus D 0 | placebo |
| D 0 | 16414 ± 4714 | | | 13281 ± 4335 | | | |
| D 28 | 14068 ± 5348 | −14.3% | *** p < 0.001 | 13525 ± 4290 | +1.8% | ns | *** p < 0.001 |
| D 56 | 13638 ± 4158 | −16.9% | *** p < 0.001 | 14045 ± 4617 | +4.8% | * p < 0.05 | *** p < 0.001 |

Thus, compared to the placebo, treatment with the active resulted in a significant reduction pigment defect of −16.1% and −21.7%, respectively, after 28 and 56 days.

For comparison, the results with ascorbic acid are shown in the following table:

| | Ascorbic acid | | | Placebo | | | Unpaired |
|---|---|---|---|---|---|---|---|
| | Mean +/− | Average | Paired | Mean +/− | Average | Paired | t test |
| Pigment | SD (arbi- | volume | t test | SD (arbi- | volume | t test | versus |
| defect | trary unit) | (mm³) vs D 0 | versus D 0 | trary unit) | (mm³) vs D 0 | versus D 0 | placebo |
| D 0 | 15660 ± 4532 | | | 12477 ± 3877 | | | |
| D 28 | 13826 ± 4311 | −11.7% | *** p < 0.001 | 12303 ± 4014 | −1.4% | ns | ** p < 0.001 |
| D 56 | 13078 ± 3731 | −16.5% | *** p < 0.001 | 12151 ± 3865 | −2.6% | # p < 0.1 | *** p < 0.001 |

### EXAMPLE 16: ULTRASOUND MEASUREMENTS USING DUB® SKIN SCANNER

### Method

The DUB Skin Scanner system is a high-frequency, high-resolution ultrasound diagnostic tool for use in dermatology, cosmetics and pharmaceuticals and clinical research. 75 MHz ultrasound is a popular method for non-invasive skin analysis. High frequency ultrasound (75 MHz) can explore the superficial layers of the skin and can penetrate up to 3 mm into the skin. Skin ultrasound is based on the population of ultrasound in the skin and on the direction and measurement of the amplitudes of the part reflected by the interfaces separating two media having different characteristics of acoustic waves. An ultrasound imaging technique used for the visualization of the structure of the subcutaneous body allows the evaluation of the homogeneity of the skin, including the density and the skin thickness.

Three measurements of thickness and density were taken to obtain an average for each image.

For this study, the analysis of the density of the dermis was carried out on D0, D28 and D56.

### Results

As can be seen from the results shown in the following table, a clear increase of dermis density was observed after 28 and 56 days of application of the active cream containing 0.4% of epigallocatechin gallate 4′-O-α-monoglucoside:

| | Active | | | Placebo | | | Unpaired |
|---|---|---|---|---|---|---|---|
| | Mean +/− | Average | Paired | Mean +/− | Average | Paired | t test |
| Dermis | SD (arbi- | volume | t test | SD (arbi- | volume | t test | versus |
| density | trary unit) | (mm³) vs D 0 | versus D 0 | trary unit) | (mm³) vs D 0 | versus D 0 | placebo |
| D 0 | 37.4 ± 6.1 | | | 40.4 ± 8.0 | | | |
| D 28 | 38.7 ± 6.4 | +3.4% | ns | 37.8 ± 8.6 | −6.5% | * p < 0.05 | ** p < 0.01 |
| D 56 | 38.8 ± 8.2 | +3.6% | ns | 36.7 ± 6.4 | −7.3% | # p < 0.1 | # p < 0.1 |

Also the application of the cream containing 2% of ascorbic acid led to a clear increase of dermis density after 28 and 56 days:

| | | | | | | | Mann |
|---|---|---|---|---|---|---|---|
| | Ascorbic acid | | | Placebo | | | Whitney |
| | Mean +/− | Average | Wilcoxon | Mean +/− | Average | Wilcoxon | test |
| Dermis | SD (arbi- | volume | test | SD (arbi- | volume | test | versus |
| density | trary unit) | (mm³) vs D 0 | versus D 0 | trary unit) | (mm³) vs D 0 | versus D 0 | placebo |
| D 0 | 37.7 ± 8.1 | | | 41.8 ± 7.8 | | | |
| D 28 | 39.1 ± 8.3 | +3.8% | # p < 0.1 | 36.4 ± 8.2 | −12.8% | ** p < 0.01 | ** p < 0.01 |
| D 56 | 40.7 ± 9.2 | +7.2% | ns | 40.0 ± 6.2 | −4.9% | ns | # p < 0.1 |

### EXAMPLE 17: AGE-SPOT REMOVAL INVESTIGATION USING RAMAN SPECTROSCOPY

### Panel Description

A double blind and placebo controlled clinical evaluation was carried out on 45 female Caucasian volunteers aged from 45 to 75 years. The volunteers were presenting brown spots on their hands. All the subjects participating in the study gave their informed consent at the beginning of the study.

The volunteers applied the products containing the purified preparation of Example 1 (containing epigallocatechin gallate 4′-O-α-monoglucoside) at 0.4% or ascorbic acid at 2% or placebo on their hands twice daily (morning and evening) for 56 days. At D0 and D56, the anti-spot efficacy was analyzed by Raman spectroscopy.

### Compositions

The compositions described in Example 12 were used.

### Raman Spectroscopy

The set-up included a confocal Raman probe coupled to a dispersive Raman spectrograph. The excitation laser beam was sent to the remote probe via a 5 μm core-diameter fiber and the Raman signal was conveyed to the spectrograph via a 100 μm core-diameter fiber. The probe was equipped with a 100× long working distance objective operating in air.

A piezo-electric device allowed to collect Z Raman profiles by assuring axial measurements from the surface down to a defined depth in the skin. A color video camera integrated in the probe permitted to visualize the skin surface. This camera is also useful to control the focalization of the laser on the skin.

The spectrograph was equipped with a CCD (Coupled Charge Detector) of 1024×256 elements cooled by Peltier effect, and an 830 grooves/mm grating, which allows to cover a large spectral range from 550 to 3700 cm⁻¹ in a single shot acquisition, with a spectral resolution of about 6 cm⁻¹. The excitation source was a 660 nm laser diode and the power at the sample was set to 30 mW in accordance with protection standards for radiation. For protection against laser radiation, protective glasses were used.

The 660 nm excitation was chosen as the optimal compromise between generation of parasitic fluorescence and sensitivity of the CCD camera over the whole spectral range. For skin characterization, the measurement of high wavenumber vibrations is important to access information on water content. For data acquisition, the device was controlled by the Labspec 5 software.

The Z profiles consist of an in-depth scanning through the skin. Raman spectra were collected at different focus points, from skin surface Z=0 μm to 30 μm (under the skin surface) with 3 μm steps.

The measurement depths are indicated in the following table with the corresponding acquisition times, leading to a time of 1 min for a complete profile.

| Depths (μm) | Acquisition time (s) | Depth (μm) | Acquisition time (s) |
|---|---|---|---|
| −10 | 4 | 12 | 4 |
| −6 | 4 | 15 | 4 |
| −3 | 4 | 18 | 4 |
| 0 | 4 | 21 | 4 |
| 3 | 4 | 25 | 4 |
| 6 | 4 | 30 | 4 |
| 9 | 4 | 0 | 4 |

In total, 519 Raman profiles were recorded: 2 profiles per condition and for each area (unstained area and brown spot area). Each profile comprises 14 spectra, resulting in recorded 7266 spectra.

These measurements were used for the evaluation of the spectroscopic marker of skin background fluorescence. This marker is associated with the degree of skin lightening: A decrease in this marker indicates a positive skin lightening effect, whereas a stability or an increase in this marker means that the product has no lightening effect on the skin.

### Data Analysis

The pre-processing of spectral data was performed using Matlab 7.2 (The MathWorks Inc., USA). Aberrant profiles (bad S/N (signal/noise) ratio, fluorescence, incomplete profile with a large zero offset, saturation . . . ) were excluded from the data base. Each non-aberrant profile was submitted to background corrections, which allowed to clean up the Raman signal of the skin. These background corrections included smoothing spectra with a 9-mm Savitzky-Golay filter, spikes correction, baseline correction and the normalization on the intensity of the entire wavenumber range with a vectorial function.

A baseline correction was not done in order to preserve the skin background fluorescence, which allows for the extraction of the skin fluorescence markers associated with the degree of skin lightening.

After these pre-processing steps, corrected data were used to determine the spectra corresponding to the Stratum corneum surface.

### Results

In the clinical study, the spectroscopic marker of skin background fluorescence was measured, which is associated with the degree of skin lightening after an application of products containing epigallocatechin gallate 4′-O-α-monoglucoside at 0.4% (purified preparation from Example 1) or ascorbic acid at 2% or placebo on the hands. The skin properties were evaluated using Raman spectroscopy after 28 and 56 days of twice daily application.

The results are shown in the following two tables:

| | Active | | Placebo | | Mann Whitney |
|---|---|---|---|---|---|
| Melanin | Mean +/− SD | TBX − TB0 | Mean +/− SD | TBX − TB0 | test versus |
| detection | (arbitrary unit) | (a.u) | (arbitrary unit) | (a.u) | placebo |
| D 0 | 53.52 ± 17.28 | | 50.8 ± 14.15 | | |
| D 28 | 43.31 ± 10.92 | −10.2 | 36.46 ± 8.07 | −14.3 | ns |
| D 56 | 33.12 ± 13.70 | −20.4 | 39.75 ± 9.53 | −11.03 | **p < 0.01 |

| | Ascorbic acid | | Placebo | | Mann Whitney |
|---|---|---|---|---|---|
| Melanin | Mean +/− SD | TBX − TB0 | Mean +/− SD | TBX − TB0 | test versus |
| detection | (arbitrary unit) | (a.u) | (arbitrary unit) | (a.u) | placebo |
| D 0 | 49.80 ± 13.13 | | 50.8 ± 14.15 | | |
| D 28 | 52.05 ± 11.23 | +2.25 | 36.46 ± 8.07 | −14.3 | *** p < 0.001 |
| D 56 | 32.28 ± 10.43 | −17.52 | 39.75 ± 9.53 | −11.03 | *p < 0.001 |

As can be seen from the above, epigallocatechin gallate 4′-O-α-monoglucoside and the placebo both led to a similar reduction of melanin detection after 28 days (−10.2 and −14.3, respectively). After 56 days, however, the reduction was much more pronounced with epigallocatechin gallate 4′-O-α-monoglucoside (−20.4), while melanin detection stayed about the same for the placebo (−11.03).

Ascorbic acid, on the other hand, even caused a slight increase in melanin detection after 28 days (+2.25 compared to D0), which was reversed after 56 days (−17.52).

Thus, it was demonstrated that at D56, epigallocatechin gallate 4′-O-α-monoglucoside led to a significant decrease of the level of the background fluorescence, i.e. melanin detection, between unspotted control skin and spotted skin in comparison to the placebo. This significant decrease of the color difference between the unspotted control skin and spotted skin shows a reduction of pigmented spots. Furthermore, it shows that the color difference between age-spots and surrounding skin was reduced, resulting in an improved homogeneity of the skin.

Finally, a significant difference between ascorbic acid and epigallocatechin gallate 4′-O-α-monoglucoside in favour of epigallocatechin gallate 4′-O-α-monoglucoside was also observed.

### EXAMPLE 18: CLINICAL EVALUATION ON AFRICAN SKIN

### Panel Description

The clinical study was carried out on 85 female volunteers aged from 19 to 47 years, which were divided in three groups: active, placebo and aglycone. The study was performed in South Africa on female volunteers with a dark skin type (phototype IV or more) and dull skin. All participants provided signed informed consent at the beginning of the study. The study was conducted according to the guidelines of the Declaration of Helsinki. Volunteers applied the product in full face for 56 day in twice daily application of cream containing 0.4% of the purified preparation from Example 1, containing epigallocatechin gallate 4′-O-α-monoglucoside at 0.1% (v/v), (“active”) or 0.01% of EGCG aglycone or no active (placebo). Skin color evolution was measured after 14, 28 and 56 days using L* parameter measurement by Chromameter®.

INCI formula

| | | EGCG | |
|---|---|---|---|
| | Active | aglycone | Placebo |
| Cetostearyl alcohol | 8% | 8% | 8% |
| Petroleum Jelly | 1% | 1% | 1% |
| Mineral oil | 1% | 1% | 1% |
| Arlacel 165 | 2% | 2% | 2% |
| Kemaben II | 0.8% | 0.8% | 0.8% |
| Water (QSP 100% w/v) | 85.58 | 85.97 | 85.98 |
| Triethanolamine (TEA) | 0.01% | 0.01% | 0.01% |
| Carbopol 980 | 0.01% | 0.01% | 0.01% |
| EPIGALLOCATECHIN GALLATE | 0.4% | — | — |
| MONOGLUCOSIDE* | | | |
| EPIGALLOCATECHIN GALLATE | — | 0.01% | — |
| AGLYCONE | | | |
| PHENOXYETHANOL | 0.5% | 0.5% | 0.5% |
| 1-2-HEXANEDIOL, CAPRYLYL | 0.3% | 0.3% | 0.3% |
| GLYCOL | | | |
| DIMETHICONE | 0.3% | 0.3% | 0.3% |
| FRAGRANCE | 0.1% | 0.1% | 0.1% |

*The purified preparation from Example 1 contains 32.6933 mmol/kg = 20.2869 g/kg of epigallocatechin gallate 4′-O-α-monoglucoside, resulting in a final concentration in the tested composition of 0.0811 g/l = 0.0081%.

### Skin Color Evaluation Using Chromameter® L* Parameter

The skin color was evaluated by means of a Chromameter CR400, which measures skin color, and the parameter L* (lightness) was recorded. The greater the value, the lighter the skin color. Depending on the test type, at least three or at least 15 readings were taken at each test sub-site for each time interval to serve as the specific pigmentation sport color measurement component of the study. Skin color was analyzed at D0, D14, D28 and D56.

### Statistical Analysis

For all studies in vivo, a Shapiro Wilk test was used to verify whether the raw data followed the Gaussian Law. In case of Normally-distributed data, the mean values were compared using either an unpaired or paired t student test. In case of non-Normally-distributed data, a Wilcoxon (paired) and Kruskal-Wallis test followed by a Mann-Whitney U (unpaired) test were used for paired data and unpaired data, respectively.

Results were considered as a significant for p<0.1 with #, p<0.05 with *, p<0.01 with ** and p<0.001 with ***.

### Results: Evaluation of Skin Color on Uneven Skin Tone Area

First, it was focused on an uneven skin area to compare the performance of each product.

It was found that the active of the present invention at 0.4% showed a significant increase of L* after 14 days, with a time-dependent effect showing +1.3%, +4.7% and +6.3%, respectively, after 14, 28 and 56 days, respectively. These increase is a result of progressive whitening activity.

Placebo and aglycone, on the other hand, showed opposite effect with a slight decrease of L*, demonstrating no whitening activity.

### Results: Evaluation of Global Skin Color Using 15 Measurements on Hemi Face

Subsequently, the whitening performance was analyzed using 15 Chromameter® measurement on hemi face.

The data showed a significant increase of L* by up to +6.8% in the presence of the active of the present invention, with significant effect after 14, 28 and 56 days.

Interestingly, aglycone and placebo did not show a similar performance and had almost no whitening effect at the tested time points.

During clinical evaluation, it was shown that the better bioaccumulation of the active of the present invention observed during the skin penetration analysis was well correlated to a better clinical whitening performance.

1. A method of preparing epigallocatechin gallate 4′-O-α-monoglucoside comprising incubating sucrose and a glucansucrase from Leuconostoc species, with epigallocatechin gallate, wherein the incubation is conducted in a medium comprising 2-propanol and water.

2. The method according to claim 1, wherein the medium comprises 5% to 20% (v/v) of 2-propanol.

3. The to according to claim 1, wherein the medium further comprises a lower diol and/or a lower triol selected from the group consisting of 1,3-propanediol, 1,2-propanediol, 1,2-butanediol, 1,3-butanediol, 1,4-butanediol, 2,3-butanediol, 2,4-butanediol, 1,2,3-propanetriol (glycerol), and mixtures thereof.

4. The method according to claim 1, wherein the medium does not comprise any dimethylsulfoxide nor any ethers of ethylene glycol (glymes).

5. The method according to claim 1, wherein epigallocatechin gallate is incubated at an initial concentration of from 5 to 50 mM.

6. The method according to claim 1, wherein the glucansucrase is a dextransucrase.

7. The method according to claim 1, wherein the concentration of the glucansucrase in the reaction mixture is from 0.4 to 4.0 U/ml.

8. The method according to claim 1, wherein the monoglucoside is formed in a ratio of at least 2:1 relative to the sum of the di- and tri-glucosides.

9. The method according to claim 1, wherein the 4′-O-α-monoglucoside is formed with a regioselectivity of at least 75% on a molar basis compared to other monoglucosides.

10. The method according to claim 1, wherein the epigallocatechin gallate 4′-O-α-monoglucoside represents at least 60% of the total glucosides formed.

11. Epigallocatechin gallate 4′-O-α-monoglucoside obtained by the method according to claim 1.

12. A method of providing a radiant skin complexion and/or age-spot removal, wherein epigallocatechin gallate 4′-O-α-monoglucoside is topically applied to the skin.

13. A method of providing a radiant skin complexion and/or age-spot removal, wherein the epigallocatechin gallate 4′-O-α-monoglucoside is formed by the method of claim 1.

14. A method according to claim 12 wherein the ratio of epigallocatechin gallate 4′-O-α-monoglucoside to other glucosides of epigallocatechin gallate is at least 2:1.

15. The method according to claim 12, wherein a cosmetic composition comprising the epigallocatechin gallate 4′-O-α-monoglucoside and a cosmetically acceptable carrier is topically applied to the skin.

16. The method according to claim 15, wherein the cosmetic composition comprises about 0.0001% to about 0.03% of the epigallocatechin gallate 4′-O-α-monoglucoside.

17. The method according to claim 15, wherein the cosmetic composition comprises one or more further cosmetic actives selected from the group consisting of lightening agents, dark spot erasers, skin tone agents, hydrating agents, moisturizers, UV protection actives, and/or anti-ageing actives.

18. The method according to claim 1, wherein the glucansucrase from Leuconostoc species is Leuconostoc mesenteroides NRRL B-512F.

19. The method according to claim 13, wherein the ratio of epigallocatechin gallate 4′-O-α-monoglucoside to other glucosides of epigallocatechin gallate is at least 2:1.
